(19) United States
(12) Patent Application Publication
(10) Pub. No.: US 2010/0179302 A1
(43) Pub. Date: Jul. 15, 2010

(54) Manufacture of Dichloropropanol

(75) Inventors: Philippe Krafft, Rhode Saint Genese (BE); Patrick Gilbeau, Braine-le-Comte (BE)

Correspondence Address: Solvay;c/o B. Ortego - IAM-NAFTA, 3333 Richmond Avenue, Houston, TX, 77098-3099, US

(73) Assignee: SOLVAY (SOCIETE ANONYME), Brussels (BE)

(21) Appl. No.: 12/663,887

(22) PCT Filed: Jun. 20, 2008
(86) PCT No.: PCT/EP2008/057876

§ 371 Date: Dec. 10, 2009

(30) Foreign Application Priority Data
Jun. 28, 2007 (FR) 07.56125

(51) Int. Cl. C07C 31/34; C07D 303/08; C08G 65/04; C07D 303/36; C09K 3/00

(52) U.S. Cl. 528/421; 568/844; 549/563; 549/553; 252/182.12

(57) ABSTRACT

Manufacture of dichloropropanol Process for manufacturing dichloropropanol wherein a glycerol-based product comprising at least one diol containing at least 3 carbon atoms other than 1,2-propanediol, is reacted with a chlorinating agent, and of products derived from dichloropropanol such as epichlorohydrin and epoxy resins. No figure.

The present patent application claims the benefit of the French Patent application FR 07/56125 filed on 28 Jun. 2007 and of the U.S. Provisional Patent Application USP 61/013710 filed on 14 Dec. 2007, the content of both of which is incorporated herein by reference.

The present invention relates to a process for manufacturing dichloropropanol, more specifically to a process for manufacturing dichloropropanol from a product based on glycerol (1,2,3-propanetriol). The invention also relates to a process for the purification of the glycerol-based product. The invention finally relates to the use of the glycerol-based product and of the purified product in the manufacture of dichloropropanol and of derived products such as epichlorohydrin and epoxy resins.

Dichloropropanol, for example, is a reaction intermediate in the manufacture of epichlorohydrin and epoxy resins (Kirk-Othmer Encyclopedia of Chemical Technology, Fourth Edition, 1992, Vol. 2, page 156, John Wiley & Sons, Inc.).

According to known processes, dichloropropanol can be obtained in particular by hypochlorination of allyl chloride, by chlorination of allyl alcohol and by hydrochlorination of glycerol.

The latter process exhibits the advantage that the dichloropropanol can be obtained starting from renewable raw materials. It is known that petrochemical natural resources, from which the fossil materials originate, for example oil, natural gas or coal, available on Earth are limited.

The process of hydrochlorination of glycerol has a selectivity which leaves much to be desired, which results in the formation of by-products that are difficult to eliminate. This requests eventually also an extensive purification of the starting raw materials.

The invention aims to solve this problem by providing a new process for manufacturing dichloropropanol, the dichloropropanol being suitable for further uses, as for example the manufacture of epichlorohydrin.

The invention therefore relates in a first embodiment to a process for manufacturing dichloropropanol wherein a glycerol-based product that comprises at least one diol containing at least 3 carbon atoms other than 1,2-propanediol, is reacted with a chlorinating agent.

One of the essential characteristics of the present invention resides in the identification of glycerol impurities which although they interfere with the operations for separation and treatment of the effluents from the processes employing glycerol, the said glycerol can nevertheless be used as raw material in the manufacture of dichloropropanol.

In the rest of the document, and except where mentioned otherwise, the term “diol” will represent a diol containing at least 3 carbon atoms other than 1,2-propanediol.

In the process according to the invention, the diol comprised in the glycerol-based product may be 1,3-propanediol, butanediol, or a mixture of the two, and is often 1,3-propanediol.

The butanediol may be 1,2-butanediol, 2,3-butanediol, 1,4-butanediol or a mixture of at least two of them. 2,3-butanediol, 1,4-butanediol and mixtures thereof are often encountered and 1,4-butanediol is encountered more often.

In the process according to the invention, the glycerol-based product may also contain, in addition, 1,2-ethanediol (ethylene glycol), 1,2-propanediol (propylene glycol), or a mixture of these two compounds. These compounds may come from the glycerol manufacturing processes such as the fermentation of mono- and polysaccharides derived from biomass or the hydrogenation of monosaccharides and derived alcohols.

In the process according to the invention, the glycerol content of the glycerol-based product is generally greater than or equal to 500 g of glycerol per kg of product, usually greater than or equal to 750 g/kg, commonly greater than or equal to 900 g/kg, in most cases greater than or equal to 950 g/kg, often greater than or equal to 990 g/kg, frequently greater than or equal to 995 g/kg, particularly greater than or equal to 999 g/kg and specifically greater than or equal to 999.5 g/kg. A glycerol content greater than or equal to 999.9 g/kg is convenient.

In the process according to the invention, the diol content of the glycerol-based product is generally less than or equal to 100 g/kg of product, usually less than or equal to 90 g/kg, commonly less than or equal to 50 g/kg, in many cases less than or equal to 10 g/kg, often less than or equal to 5 g/kg, frequently less than or equal to 1 g/kg, particularly less than 0.7 g/kg, specifically less than or equal to 0.5 g/kg and often less than or equal to 0.2 g/kg. This amount is usually greater than or equal to 0.001 g/kg, generally greater than or equal to 0.005 g/kg, commonly greater than or equal to 0.01 g/kg, in many cases greater than or equal to 0.04 g/kg and particularly greater than or equal to 0.1 g/kg.

In the process according to the invention, the ethylene glycol content of the glycerol-based product is generally less than or equal to 100 g/kg of product. This content is usually less than or equal to 90 g/kg, commonly less than or equal to 50 g/kg, in many cases less than or equal to 10 g/kg, particularly less than or equal to 1 g/kg, specifically less than or equal to 0.5 g/kg and often less than or equal to 0.2 g/kg. This amount is usually greater than or equal to 0.001 g/kg of product, generally greater than or equal to 0.005 g/kg, commonly greater than or equal to 0.01 g/kg, in many cases greater than or equal to 0.04 g/kg and often greater than or equal to 0.1 g/kg.

In the process according to the invention, the propylene glycol content of the glycerol-based product is generally less than or equal to 100 g/kg of product. This content is usually less than or equal to 90 g/kg, commonly less than or equal to 50 g/kg, in many cases less than or equal to 10 g/kg, particularly less than or equal to 1 g/kg, specifically less than or equal to 0.5 g/kg and often less than or equal to 0.2 g/kg. This amount is usually greater than or equal to 0.001 g/kg of product, generally greater than or equal to 0.005 g/kg, in many cases greater than or equal to 0.01 g/kg, commonly greater than or equal to 0.04 g/kg and often greater than or equal to 0.1 g/kg.

In the process according to the invention, the glycerol-based product generally contains glycerol alkyl ethers such as described in Application WO2007/144335 in the name of Solvay SA, more specifically from page 2, line 6 to page 3, line 25, and the content of which is incorporated here by reference.

The content of glycerol alkyl ethers of the glycerol-based product is generally less than or equal to 90 g/kg of product, usually less than or equal to 50 g/kg, in many cases less than or equal to 10 g/kg, commonly less than or equal to 5 g/kg, particularly less than or equal to 1 g/kg, specifically less than or equal to 0.5 g/kg and often less than or equal to 0.2 g/kg. This amount is usually greater than or equal to 0.001 g/kg, generally greater than or equal to 0.005 g/kg, commonly greater than or equal to 0.01 g/kg, in many cases greater than or equal to 0.04 g/kg and often greater than or equal to 0.1 g/kg.

The glycerol alkyl ethers are generally selected from methyl, ethyl, propyl, butyl ethers and any mixtures of at least two of them, usually from methyl ethers, and in many cases from monomethyl ethers. Such ethers are for examples 2-methoxy-1,3-propanediol, 3-methoxy-1,2-propanediol, and mixtures thereof.

In the process according to the invention, the glycerol-based product may also comprise monoalcohols, such as the monoalcohols described in Application WO2007/144335 in the name of Solvay SA, more specifically from page 3, lines 26 to 31, and the content of which is incorporated here by reference.

These monoalcohols can be present in an amount generally greater than or equal to 0.001 g/kg of product, usually greater than or equal to 0.01 g/kg, commonly greater than or equal to 0.1 g/kg, in many cases greater than or equal to 0.5 g/kg, and often greater than or equal to 1 g/kg. This amount is generally less than or equal to 20 g/kg of product, usually less than or equal to 10 g/kg, commonly less than or equal to 5 g/kg and in many cases less than or equal to 2 g/kg.

These monoalcohols are usually selected from methanol, ethanol, n-propanol, isopropanol, butanol, and any mixture of at least two of them, generally from methanol, ethanol, and mixtures thereof, and the monoalcohol is often methanol.

In the process according to the invention, the glycerol-based product may also comprise water in an amount generally greater than or equal to 0.01 g/kg of product and less than or equal to 100 g/kg. This amount is usually less than or equal to 50 g/kg, commonly less than or equal to 40 g/kg, in many cases less than or equal to 30 g/kg, particularly less than or equal to 20 g/kg, specifically less than or equal to 10 g/kg, often less than or equal to 5 g/kg and frequently less than or equal to 2 g/kg. This amount is usually higher than or equal to 0.05 g/kg, commonly higher than or equal to 0.1 g/kg, in many cases higher than or equal to 0.5 g/kg, and particularly higher than or equal to 1 g/kg.

In the process according to the invention, the glycerol-based product according to the invention may also comprise ketones, aldehydes, alkyl esters of fatty acids, glycerol esters, carboxylic acids, glycerol oligomers and salts such as described in Application WO2007/144335 in the name of Solvay SA, more specifically, from page 5, lines 12 to 20, and the content of which is incorporated here by reference.

The ketone can be selected from acetone, hydroxyacetone and dihydroxyacetone.

These ketones are generally present in an amount greater than or equal to 0.001 g/kg of product and usually greater than or equal to 0.005 g/kg. This amount is generally less than 10 g/kg of product, usually less than or equal to 5 g/kg of product, commonly less than or equal to 1 g/kg of product, in many cases less than or equal to 0.5 g/kg of product, particularly less than or equal to 0.1 g/kg of product and specifically less than or equal to 0.01 g/kg of product.

The aldehyde can be selected from acetaldehyde, propionaldehyde, butyraldehyde, acrolein, glyceraldehyde and any mixture of at least two of them.

These aldehydes are generally present in an amount greater than or equal to 0.001 g/kg of product and usually greater than or equal to 0.005 g/kg. This amount is generally less than 10 g/kg of product, usually less than or equal to 5 g/kg of product, commonly less than or equal to 1 g/kg of product, in many cases less than or equal to 0.5 g/kg of product, particularly less than or equal to 0.1 g/kg of product and specifically less than or equal to 0.01 g/kg of product.

The esters can be selected from alkyl esters of fatty acids, glycerol esters, such as, for example, mono- and diglycerides, and any mixture thereof. Alkyl esters of fatty acids may be selected from alkyl palmitate, alkyl oleate, alkyl linoleate, alkyl stearate, and any mixture of at least two of them. Methyl esters are more often encountered. Glycerol esters may be selected from glycerol acetate, glycerol mono palmitate, mono oleate, mono linoleate, mono linolenate, mono stearate and any mixture of at least two of them.

These esters are generally present in an amount greater than or equal to 0.001 g/kg of product, usually greater than or equal to 0.1 g/kg, commonly greater than or equal to 0.5 g/kg, in many cases greater than or equal to 1 g/kg and often greater than or equal to 2 g/kg. This amount is generally less than 50 g/kg of product, usually less than or equal to 30 g/kg of product, commonly less than or equal to 20 g/kg of product, in many cases less than or equal to 15 g/kg of product, particularly less than or equal to 10 g/kg of product and specifically less than or equal to 5 g/kg of product.

The carboxylic acids are usually fatty acids. They can be selected from palmitic, oleic, linoleic, linolenic, stearic acid, and any mixture of at least two of them.

These acids are generally present in an amount greater than or equal to 0.001 g/kg of product, usually greater than or equal to 0.1 g/kg, commonly greater than or equal to 0.5 g/kg, in many cases greater than or equal to 1 g/kg and often greater than or equal to 2 g/kg. This amount is generally less than 50 g/kg of product, usually less than or equal to 30 g/kg of product, commonly less than or equal to 20 g/kg of product, in many cases less than or equal to 15 g/kg of product, particularly less than or equal to 10 g/kg of product and specifically less than or equal to 5 g/kg of product.

In the process according to the invention, the glycerol-based product according to the invention may also comprise glycerol oligomers, such as described in Patent Application FR 08/52206 in the name of Solvay SA, more specifically from page 2, line 6 to page 3, line 20, and the content of which is incorporated here by reference.

By glycerol oligomers, one intends to denote compounds resulting from condensation reactions between at least two glycerol molecules.

In the process according to the invention the glycerol oligomer which can be present in the glycerol-based product results generally from condensation reactions between at least two glycerol molecules (dimer of glycerol) and at most seven glycerol molecules (heptamer of glycerol), usually at most six glycerol molecules (hexamer of glycerol), commonly at most five glycerol molecules (pentamer of glycerol), in many cases at most four glycerol molecules (tetramer of glycerol), and often at most three glycerol molecules (trimer of glycerol).

The glycerol oligomer is frequently a compound resulting from the condensation of two glycerol molecules.

The glycerol oligomer can be selected from oligomers exhibiting a linear structure, a branched structure, a cyclic structure, and any mixture of at least two of them.

By oligomers exhibiting a linear structure, one intends to denote oligomers in the molecule of which all the carbon atoms are located in only one atoms chain, which does not constitute a cycle.

By oligomers exhibiting a branched structure, one intends to denote oligomers in the molecule of which the carbon atoms are located in at least two atoms chains.

By oligomers exhibiting a cyclic structure, one intends to denote oligomers in the molecule of which at least one part of the carbon atoms are located in at least one cycle. The number of carbon atoms constituting the cycle is generally higher than or equal to 6, usually higher than or equal to 7, and commonly higher than or equal to 8. The number of carbon atoms constituting the cycle is generally lower than or equal to 20, usually lower than or equal to 15, and commonly lower than or equal to 10. The cycle comprises generally at least 2 oxygen atoms. The oligomers of glycerol with a cyclic structure, comprising only one cycle containing 6, 7 or 8 atoms, of which 2 atoms are oxygen atoms are often encountered.

The glycerol oligomer is often chosen from dimers of glycerol, trimers of glycerol, tetramers of glycerol, and any mixture of at least two of them. The glycerol oligomer is often a dimer of glycerol.

The dimer of glycerol is generally a mixture of dimers of glycerol, comprising one dimer with a linear structure, at least one dimer with a branched structure and at least one dimer with a cyclic structure. The dimer with the cyclic structure comprises generally at least one cycle and usually only one cycle. That cycle comprises generally 6 atoms, usually 7 atoms and commonly 8 atoms, of which 2 atoms are oxygen atoms, the others atoms being carbon atoms.

The glycerol oligomer is generally a mixture of 3-(2,3-dihydroxy-propoxy)-propane-1,2-diol, 3-(2-hydroxy-1-hydroxymethyl-ethoxy)-propane-1,2-diol, 2-(2-hydroxy-1-hydroxymethyl-ethoxy)-propane-1,3-diol), of cis- and trans-2,5-bis-hydroxymethyl-1,4-dioxane, of cis- et trans-2,6-bis-hydroxymethyl-1,4-dioxane, de cis-et trans-6-hydroxy-2-hydroxymethyl-1,4-dioxepane, and of cis-et trans-3,7-dihydroxy-1,5-dioxocane.

The glycerol oligomer is usually a glycerol oligomer with a cyclic structure.

The glycerol oligomer can be selected from dimers of glycerol with a linear structure, dimers of glycerol with a branched structure, and any mixture thereof.

In the following description, glycerol oligomers will also be named polyglycerols and dimers, trimers and tetramers of glycerol will also be named diglycerols, triglycerols and tetraglycerols.

In the glycerol based product used in the process according to the invention, the polyglycerol content is generally lower than or equal to 10 g/kg of glycerol-based product, usually lower than or equal to 5 g/kg, commonly lower than or equal to 2.5 g/kg, in many cases lower than or equal to 1 g/kg, particularly lower than or equal to 0.1 g/kg. This content is usually higher than or equal to 0.05 g/kg.

In the glycerol based product used in the process according to the invention, the diglycerol content is generally lower than or equal to 10 g/kg of glycerol-based product, usually lower than or equal to 5 g/kg, commonly lower than or equal to 2.5 g/kg, in many cases lower than or equal to 1 g/kg, particularly lower than or equal to 0.1 g/kg. This content is usually higher than or equal to 0.05 g/kg.

In the glycerol based product used in the process according to the invention, the content of 3-(2,3-dihydroxy-propoxy)-propane-1,2-diol is generally lower than or equal to 10 g/kg of glycerol-based product, usually lower than or equal to 5 g/kg, commonly lower than or equal to 2.5 g/kg, in many cases lower than or equal to 1 g/kg, particularly lower than or equal to 0.1 g/kg. This content is usually higher than or equal to 0.05 g/kg.

In the glycerol based product used in the process according to the invention, the sum of the contents of 3-(2-hydroxy-1-hydroxymethyl-ethoxy)-propane-1,2-diol and 2-(2-hydroxy-1-hydroxymethyl-ethoxy)-propane-1,3-diol is generally lower than or equal to 10 g/kg of glycerol-based product, usually lower than or equal to 5 g/kg, commonly lower than or equal to 2.5 g/kg, in many cases lower than or equal to 1 g/kg, particularly lower than or equal to 0.1 g/kg. This content is usually higher than or equal to 0.05 g/kg.

In the glycerol based product used in the process according to the invention, the sum of the contents of cis- et trans-2,5-bis-hydroxymethyl-1,4-dioxane, cis- et trans-2,6-bis-hydroxymethyl-1,4-dioxane, cis- et trans-6-hydroxy-2-hydroxymethyl-1,4-dioxepane, et cis- et trans-3,7-dihydroxy-1,5-dioxocane is generally lower than or equal to 10 g/kg of glycerol-based product, usually lower than or equal to 5 g/kg, commonly lower than or equal to 2.5 g/kg, in many cases lower than or equal to 1 g/kg, particularly lower than or equal to 0.1 g/kg. This content is usually higher than or equal to 0.05 g/kg.

In the glycerol based product used in the process according to the invention, the salts can be chosen from alkaline metal chlorides, alkaline metal sulfates, alkaline metal phosphates, alkaline-earth metal chlorides, alkaline-earth metal sulfates, alkaline-earth metal phosphates, and any mixtures of at least two of them.

The alkaline metal is generally sodium, usually potassium and commonly a mixture thereof. The alkaline-earth metal is generally calcium, usually magnesium and commonly a mixture thereof. Mixtures of sodium and potassium chlorides and/or sodium and potassium sulfates are generally encountered.

The salt can be present in an amount generally greater than or equal to 0.0005 g/kg of product, usually greater than or equal to 0.001 g/kg and commonly greater than or equal to 0.01 g/kg. This amount is generally less than or equal to 10 g/kg of product, usually less than or equal to 1 g/kg of product, and commonly less than or equal to 0.1 g/kg of product.

The glycerol-based product used in the process for manufacturing dichloropropanol according to the invention may contain a nitrogen compound.

In the process according to the invention, the glycerol-based product according to the invention may also comprise contain a nitrogen compound, such as described in Patent Application FR 07/59891 in the name of Solvay SA, more specifically from page 2, line 17 to page 3, line 20, and the content of which is incorporated here by reference.

Molecular nitrogen is not considered as a nitrogen compound.

The nitrogen compound can be an inorganic or an organic compound. By inorganic compound, one intends to designate compound the molecule of which does include neither a carbon-carbon bond, nor a carbon-hydrogen bond. By organic compound, one intends to designate a compound the molecule of which includes at least one carbon-carbon bond or one carbon-hydrogen bond.

The nitrogen inorganic compound can be selected from ammonia, hydrazine, chlroramines, ammonium inorganic salts, nitrates, nitrites, cyanates, isocyanates, isothiocyantes of metals or ammonium, and any mixture of at least two of them.

The nitrogen organic compound can be selected from nitrogen compounds present in cells of plant origin, generally among amines, urea, proteins, peptides, aminoacids, glucosinolates and their degradation products (isothiocyantes, thiocyanates, nitriles, oxazolidinethiones), phospholipids containing nitrogen, chlorophyll, sinapine, and any mixtures of at least two of them.

Examples of phospholipids containing nitrogen are phospatidyl chloline, phosphatidyl serine and phosphatidyl ethanolamine.

Aminoacids free or able to be included in peptides or proteins cane be selected from alanine, arginin, aspartic acid, cystein, glutamic acid, glycin, histidin, isoleucin, leucin, lysin, methionin, phenylalanin, prolin, serin, threonin, trytptophane, tyrosin, valin, and any mixture of at least two of them.

Glucosinolates can be selected from sinigri, gluconapin, glucobrassicanapin, glycorucin, glucoberteroin, glucoraphanin, glucoalyssin, gluconasturtiin, progoitrin, napoleiferin, glucobrassicin, neoglucobrassicin, and any mixture of at least two of them.

In the glycerol based product used in the process according to the invention, the total content of nitrogen compound expressed as elemental nitrogen is generally lower than or equal to 1 g of N/kg, usually lower than or equal to 0.5 g of N/kg, commonly lower than or equal to 0.1 g of N/kg, in many cases lower than or equal to 0.05 g of N/kg, particularly lower than or equal to 0.03 g of N/kg and specifically lower than or equal to 0.01 g of N/kg. This content is usually higher than or equal to 0.1 mg N/kg.

The glycerol based product used in the process according to the invention can also contain metallic and non-metallic elements chosen from Al, As, Be, Ca, Cd, Co, Cr, Cu, Fe, Hg, Na, Ni, P, Pb, S, Sb, Se, Sn, Te, Ti, V, Zn, and any mixture thereof. The total content of those elements is usually lower than or equal to 0.05 g/kg, generally lower than or equal to 0.035 g/kg, commonly lower than or equal to 0.025 g/kg, in many cases lower than or equal to 0.015 g/kg and particularly lower than or equal to 0.005 g/kg. That total content is usually higher than or equal to 0.1 mg/kg.

The glycerol alkyl ethers, monoalcohols, water, alkyl esters of fatty acids, fatty acids, glycerol esters, glycerol oligomers and salts may be by-products of glycerol manufacturing processes such as, for example, the processes for conversion of oils or fats of vegetable or animal origin via transesterification, saponification or hydrolysis reactions.

The glycerol-based product used in the process for manufacturing dichloropropanol according to the invention may be manufactured by conversion of saccharides and polysaccharides derived from biomass such as, for example, fermentation and/or hydrogenation processes, carried out under conditions such that the diol was formed and was not separated from the glycerol. Biomass comprises harvest products, trees, grass, harvest residues, forest residues, animal waste and solid municipal waste.

The invention therefore also relates to a process for manufacturing a glycerol-based product comprising at least one diol containing at least 3 carbon atoms except for 1,2-propanediol, in which a saccharide or a polysaccharide is reacted with a fermentation agent under conditions such that the diol is formed and is not separated from the glycerol.

Alternatively, the glycerol-based product used in the process for manufacturing dichloropropanol according to the invention may also be obtained when glycerol is heated under a pressure greater than or equal to 1 bar absolute, at a temperature greater than or equal to 150° C., in the presence of a reducing agent, such as a metal sulphite or phosphite for example.

The invention also relates to a process for manufacturing the glycerol-based product used in the process for manufacturing dichloropropanol according to the invention, wherein the glycerol-based product comprising at least one diol containing at least 3 carbon atoms except for 1,2-propanediol is subjected to a heating operation under a pressure greater than or equal to 1 bar absolute, at a temperature greater than or equal to 150° C., in the presence of a reducing agent.

The glycerol may have been obtained starting from fossil raw materials and/or renewable raw materials, preferably starting from renewable raw materials, such as defined in Application WO 2006/100312 in the name of Solvay SA, from page 4, line 18 to page 5, line 24, the content of which is incorporated here by reference. Glycerol obtained starting from renewable raw materials is, for example, glycerol obtained in processes for conversion of animal or vegetable oils and/or fats, such as hydrolysis, saponification, transesterification, aminolysis or hydrogenation processes and enzymatic rupture processes, such as transesterification or hydrolysis with lipase-type enzymes, such as described in “Medium and Long-Term Opportunities and Risks of the Biotechnological Production of Bulk Chemicals from Renewable Resources, The Potential of White Biotechnology, The BREW Project, Final report Prepared under the European Commission's GROWTH Programme (DG Research), Utrecht, September 2006, pp. 29-31”. Glycerol obtained starting from renewable raw materials is, for example, glycerol obtained in processes for conversion of mono- and polysaccharides and derived alcohols, such as fermentation, and thermochemical process such as hydrogenation and hydrogenolysis, as described in “Industrial Bioproducts: Today and Tomorrow, Energetics, Incorporated for the U.S. Department of Energy, Office of Energy Efficiency and Renewable Energy, Office of the Biomass Program, July 2003, pages 49, 52 to 56”. The mono- and polysaccharides, such as for example, starch, cellulose and hemicellulose may themselves be obtained from biomass.

The invention also relates to a process for the purification of the glycerol-based product in which the product is subjected to at least one treatment, optionally under reduced pressure, chosen from evaporative concentration, evaporative crystallization, distillation, fractional distillation, stripping or liquid/liquid extraction operations.

The term “evaporative concentration” is intended to denote a process of partial evaporation of the product which makes it possible to concentrate the residual product to less volatile entities. The term “evaporative crystallization” is intended to denote a process resulting in the crystallization of a compound by removing, by evaporation, a compound which promotes its dissolution in the medium. These processes are described in “Perry's Chemical Engineers' Handbook” in the 11th section of the 7th edition.

The term “distillation” is intended to denote the type of separation conventional in chemical engineering and described, for example, in “Perry's Chemical Engineers' Handbook” in the 13th section of the 7th edition.

The term “fractional distillation” is understood to mean a series of distillations where the distillate is withdrawn batchwise.

The term “stripping” is intended to denote the separation of a substance by entrainment using the vapour of a pure material. In the process according to the invention, this material can be any compound which is inert with respect to glycerol, such as, for example, steam, air, nitrogen and carbon dioxide.

The term “liquid/liquid extraction” is understood to mean bringing into contact with an appropriate completely or partially immiscible solvent which makes it possible to selectively extract the desired compounds, optionally according to a countercurrent process, such as described in “Perry's Chemical Engineers' Handbook” in the 15th section of the 7th edition.

The stripping, evaporative concentration, evaporative crystallization, liquid/liquid extraction and distillation treatments may be combined, for example in a stripping column surmounted by a distillation section or in a partial evaporator supplying a distillation column or by combining a liquid/liquid extraction, stripping of the residual solvent contained in the glycerol-enriched stream and distillation of the solvent enriched with extracted compounds.

The diols, monoalcohols and the glycerol alkyl ethers are recovered in the distilled, evaporated or stripped fraction and the purified glycerol-based product constitutes an intermediate cut from the distillation, evaporation or stripping treatment.

When the treatment consists of a partial evaporation of the product, the temperature of the glycerol-rich zone is generally greater than or equal to 0° C., often greater than or equal to 80° C. and frequently greater than or equal to 100° C. This temperature is generally less than or equal to 280° C., often less than or equal to 250° C., and frequently less than or equal to 200° C. The temperature in the glycerol-depleted zones is generally greater than or equal to −20° C., preferably greater than or equal to −10° C., particularly preferably greater than or equal to 0° C. This temperature is generally at most equal to the temperature of the glycerol-rich zone, preferably at least 5° C. below this temperature, particularly preferably at least 10° C. below this temperature.

When the treatment is carried out by liquid/liquid extraction, the temperature is generally greater than or equal to 20° C., preferably greater than or equal to 40° C., more particularly greater than or equal to 50° C. This temperature is generally less than or equal to 200° C., preferably less than or equal to 150° C. and more particularly preferably less than or equal to 120° C.

The treatment pressure is generally greater than or equal to 0.001 mbar. This pressure is generally less than or equal to 1 bar, often less than or equal to 0.5 bar, frequently less than or equal to 0.3 bar and more specifically less than or equal to 0.25 bar. When the treatment comprises a separate evaporation step, the latter is generally carried out at a pressure less than or equal to 2 bar absolute, preferably at a pressure less than or equal to 1 bar absolute, particularly preferably at a pressure less than or equal to 0.5 bar absolute. It is generally carried out at a pressure greater than or equal to 0.1 mbar, preferably at a pressure greater than or equal to 0.2 mbar. When the evaporation step is combined with a distillation or fractional distillation step, it is carried out at a pressure at least equal to the pressure of the step carried out at the lowest pressure, preferably at a pressure at least 10 mbar greater than the pressure of the step carried out at the lowest pressure. The stripping step is generally carried out at a pressure less than or equal to 5 bar, preferably less than or equal to 2 bar.

In the distillation treatments with or without stripping, the reboiler ratio is generally greater than or equal to 1%, often greater than or equal to 5% and frequently greater than or equal to 10%. This reboiler ratio is less than or equal to 99% and often less than or equal to 50%. The expression “ reboiler ratio” is understood to mean, for a continuous distillation, the flow of the vaporized fraction to the reboiler over the flow of the residue.

The expression “reboiler ratio” is understood to mean, for a batchwise, fractional distillation, the ratio of the amount vaporized relative to the final residue.

The amount of the fraction distilled is generally less than or equal to 300 g/kg, often less than or equal to 100g/kg of the glycerol-based product.

The distillation, fractional distillation or stripping treatment may be preceded or followed by an operation which may, for example, be a settling, centrifugation, filtration, adsorption or ion-exchange operation. When it is a settling operation, the operation can be improved by passing through a coalescer. The adsorption operation is preferably an operation for adsorption on activated carbon.

After the treatment, a purified glycerol-based product that comprises at least one diol containing at least 3 carbon atoms other than 1,2-propanediol is obtained, in an amount generally less than or equal to 1 g/kg of purified product. This amount is usually less than 0.7 g/kg, commonly less than or equal to 0.5 g/kg, in many cases less than or equal to 0.1 g/kg of purified product, specifically less than or equal to 0.01 g/kg and particularly less than or equal to 0.001 g/kg. This content is generally greater than or equal to 0.01 mg/kg.

The invention also relates to a glycerol-based product that comprises at least one diol containing at least 3 carbon atoms other than 1,2-propanediol. The amount of the diol is usually less than 0.7 g of diol/kg of product. This amount is commonly less than or equal to 0.5 g/kg, in many cases, in many cases less than or equal to 0.1 g/kg of product, specifically less than or equal to 0.01 g/kg and particularly less than or equal to 0.001 g/kg. This amount is generally greater than or equal to 0.01 mg/kg. This product may be obtained by the purification process described above.

The diol may be 1,3-propanediol, butanediol, or a mixture of the two, and is frequently 1,3-propanediol.

The purified glycerol-based product can also contain, in addition, at least one compound selected from glycerol alkyl ethers, monoalcohols, water, ketones, aldehydes, alkyl esters of fatty acids, glycerol esters, carboxylic acids, glycerol oligomers, salts, compounds containing nitrogen, metallic elements, non-metallic elements, and any mixture of at least two of them. The nature of those compounds and their content in the purified glycerol-based product are as described above for the glycerol-based product.

The invention also relates to a process for manufacturing dichloropropanol starting from glycerol, in which a glycerol-based product that comprises at least one diol containing at least 3 carbon atoms other than 1,2-propanediol is subjected to at least one treatment, optionally under reduced pressure, chosen from evaporative concentration, evaporative crystallization, distillation, fractional distillation, stripping or liquid/liquid extraction operations, so as to reduce the diol content and to obtain a purified product which is reacted with a chlorinating agent.

The chlorinating agent generally comprises hydrogen chloride. The hydrogen chloride can be gaseous hydrogen chloride, an aqueous hydrogen chloride solution or a mixture of the two.

The chlorinating agent is such as described in Patent Applications WO 2007/144335, from page 12, line 18 to page 13, line 35, WO 2005/054167, from page 4, line 32 to page 5, line 19, and WO 2006/106153, from page 2, line 10 to page 3, line 20, in the name of Solvay SA, the contents of which are incorporated here by reference.

The reaction with the chlorinating agent may be carried out in the presence of a catalyst, preferably a carboxylic acid or a carboxylic acid derivative, such as described in Patent Application WO 2005/054167, from page 6, line 24 to page 7, line 35 in the name of Solvay SA, and in Application WO 2006/020234, from page 8, line 24 to page 9, line 10, and from page 13, line 1 to page 18, line 3, the contents of which are incorporated here by reference.

The chlorination reaction is preferably carried out in a liquid reaction medium.

The chlorination reaction may be carried out in the presence of a solvent.

The dichloropropanol formed can be separated from the other constituents of the reaction medium by any separation treatment, for example by distillation, stripping, extraction or adsorption. After this treatment, the other constituents of the reaction medium can be subjected to additional separation treatments, such as, for example, a filtration where fatty acid salts can be separated.

When the separation treatment is a distillation and when a glycerol-based product according to the process of the invention is used for manufacturing dichloropropanol, the dichloropropanol separated can be contaminated by various isomers of chloropropanol, chlorobutanol, dichloropropane and/or dichlorobutanes. These isomers can be selected from chloropropanols, dichloropropanes, and any mixtures thereof. These isomers can be selected from 3-chloro-1-propanol, 1,3-dichloropropane, and any mixture thereof. These isomers of chloropropanol, chlorobutanol, dichloropropane and/or dichlorobutanes may have boiling points close to the dichloropropanol and/or form azeotropes with water that have a boiling point close to that of the dichloropropanol/water azeotrope, making it difficult to separate them from the dichloropropanol. The treatment of the glycerol-based product used in the process for manufacturing dichloropropanol according to the invention exhibits the advantage of reducing the contamination of the dichloropropanol by these isomers.

The invention also relates to a process for manufacturing epichlorohydrin in which dichloropropanol, at least one part of which is obtained in the process for manufacturing dichloropropanol according to the invention, is subjected to a dehydrochlorination reaction. In the process for manufacturing epichlorohydrin, another part of the dichloropropanol may be obtained by a process other than glycerol chlorination. This process may be chosen from allyl chloride hypochlorination, allyl alcohol chlorination processes, and any combination thereof.

When the dichloropropanol is contaminated by various isomers of chloropropanol, chlorobutanol, dichloropropane and/or dichlorobutane, the epichlorohydrin may be contaminated by dichloropropanes and/or dichlorobutanes, usually by dichloropropanes and commonly by 1,3-dichloropropane. These dichloropropanes, and in particular 1,3-dichloropropane, have boiling points very close to that of epichlorohydrin and it is therefore very difficult to separate them from epichlorohydrin. This contamination by chlorinated impurities can be particularly troublesome, for example, when epichlorohydrin is used in applications requiring the absence of hydrolysable chloro compounds such as the manufacture of epoxy resins for the electronics industry. This contamination can be reduced by using a dichloropropanol manufactured according to the process of the invention, in particular wherein the purified glycerol-based product described above is used.

The invention finally relates to a process for manufacturing epoxy derivatives such as epoxy resins, glycidyl ethers, glycidyl esters, glycidyl amides and imides, of products which will be used in food and drink applications such as coagulants and wet-strength resins, of cationization agents, of flame retardants, of products which will be used as detergent ingredients, and of epichlorohydrin elastomers, in which epichlorohydrin, at least one part of which was obtained in the process for manufacturing epichlorohydrin according to the invention, is used. The epichlorohydrin is usually subjected to a reaction with at least one compound containing at least one active hydrogen atoms. These compounds comprise polyphenols, monoamines and diamines, aminophenols, heterocyclic imides and amides, diols including ethylene glycol and propylene glycol and aliphatic polyols, and fatty acid dimers. In the process for manufacturing epoxy derivatives such as epoxy resins, glycidyl ethers, glycidyl esters, glycidyl amides and imides, of products which will be used in food and drink applications such as coagulants and wet-strength resins, of cationization agents, of flame retardants, of products which will be used as detergent ingredients, and of epichlorohydrin elastomers, another part of the epichlorohydrin may be obtained by a process other than the dehydrochlorination of dichloropropanol derived from the chlorination of glycerol, such as a process for dehydrochlorination of dichloropropanol derived from hypochlorination of allyl chloride, a process for dehydrochlorination of dichloropropanol derived from chlorination of allyl alcohol, or an allyl chloride epoxidation process.

The epoxy resins thus obtained may be used in coating applications and in structural applications. The coating applications relate to the fields of maritime transport and of industrial maintenance (anticorrosion primer paints for metal and concrete structures), of coatings for metallic containers (food preserves, cans for drinks, drums, buckets and aerosol bottles) and for windings, of motor vehicle coatings (primers), of inks and masks for electronic circuits. The structural applications relate to the fields of structural composites (epoxy resin composites with glass, boron, carbon and aromatic polyamide fibres), of civil engineering, of floor covering (paints for coating floors, parquet, paving, tiling, self-levelling coatings, roughcast floors, tempered floors, floor coverings for cold rooms), of construction, of electrical equipment (sealing of electrical and electromechanical devices such as battery housings, resistors, fuses, thermal circuit breakers, cable joints, windings) and of electronic equipment (coatings and laminated sheets for printed circuits and encapsulation of printed circuits), of adhesives (bonding of different materials such as metals, glass, ceramics, wood, concrete, plastics) and of tooling (prototypes, master patterns, moulds and other parts) for the aerospace, automotive, foundry and maritime construction industries.

Epoxy resins also find applications in the fields of energy (wind energy), of aeronautics (honeycomb sandwich panels, helicopter rotor blades, cowls and engine nacelles, flaps, ailerons, rudders) and of fluid (gas, oil) transport.

Glycidyl esters and ethers are generally used for applications such as coatings, adhesives and reactive diluents.

Glycidyl amines and imides are usually used for applications such as outdoor powder coatings with polyesters, or in applications in which a non-yellowing epoxy resin is desirable.

Coagulants can be used for treatment of raw water for conversion to drinking water, for recycling paper of water in Pulp & Paper Industry, for paint detackification, for breaking oil emulsions, for oil and grease removal, and for sludge dewatering. They can also be used for sugar refining.

Wet-strength resins can be used in papers that will get wet such as paper towels, tea bags, coffee filters, milk cartons, meat wrapping, and wallpaper. They can also be used in the production of high fructose corn syrup and to prevent wool from shrinking.

Cationization agents are mainly used in the cationization of starch to be utilized by the paper industry for processing of high quality paper grades or for cationization of textile for dye fixing.

Flame retardants are usually used to inhibit the evolution of combustible gases in various materials such as polymers, in particular in polyurethane foams.

Examples of detergent ingredients are surfactants or surface deposition enhancing materials. They are usually used as components of cleaning compositions for instance dishwashing, laundry compositions, shampoos and synbars.

The epichlorohydrin elastomers are generally used in specialty applications, like for instance automotive components (fuel pump diaphragms, emission control hoses, motor mounts, gaskets, seals and portable fuel tanks), in the aircraft industry, for specialty roofing membranes, coated fabrics, solvent storage containers, paper mill and printing roll and in a variety of oil specialties.

The examples below are intended to illustrate the invention without, however, limiting it.

### EXAMPLE 1

A reactor has been fed continuously with glycerol containing 1.5 g of 1,3-propanediol per kg and 30 g of adipic acid per kg, at a rate of 14.3 g/h/l of reactor, with gaseous hydrogen chloride at rate of 11.3 g/h/l of reactor. The reactor has been operated at a temperature of 117° C. and at a pressure of 1.14 bar. The reactor was surmounted by a distillation column from which dichloropropanol containing water has been withdrawn. The liquid reaction mixture has been withdrawn from the reactor and has been sent to a distillation unit to recover dichlororopanol.

The dichloropropanol which has been recovered from both withdrawals contained 1.67 g of 3-chloro-1-propanol and 0.44 g of 1,3-dichloropropane per kg of dichloropropanol.

### EXAMPLE 2

The procedure of example 1 has been followed except that the glycerol contained 0.005 g of 1,3-propanediol per kg.

A reactor has been fed continuously with glycerol containing 0.005 g of 1,3-propanediol per kg and 12 g of adipic acid per kg, at a rate of 21.5 g/h/l of reactor, with gaseous hydrogen chloride at rate of 17 g/h/l of reactor. The reactor has been operated at a temperature of 119° C. and at a pressure of 1.19 bar. The reactor was surmounted by a distillation column from which dichloropropanol containing water has been withdrawn. The liquid reaction mixture has been withdrawn from the reactor and has been sent to a distillation unit to recover dichlororopanol.

The dichloropropanol which has been recovered from both withdrawals contained 0.011 g of 3-chloro-1-propanol and less than 0.010 g of 1,3-dichloropropane per kg of dichloropropanol.

### EXAMPLE 3

A well stirred reactor has been fed continuously with dichloropropanol containing 0.223 g of 1,3-dichloropropane and 2.93 g of 3-chloro-1-propanol per kg of dichloropropanol at a rate of 5.07 kg/h/l of reactor, with a sodium hydroxide solution containing 500 g of NaOH per kg at rate of 2.83 kg/h/l of reactor and with recycled water at a rate of 6.23 kg/h/l of reactor. The reactor has been operated at a temperature of 45° C. and at a pressure of 1 bar. The reactor was fitted with an overflow system for withdrawal of the liquid reaction medium. The liquid reaction medium was biphasic. The lighter phase which contained the salt and a small quantity of organics has been stripped with steam and the condensate has been recycled to the reactor. The heavier phase which contained the major part of the epichlorohydrin formed in the reactor has been submitted continuously to a first distillation operation under vacuum in a packed column to separate epichlorohydrin, water and the low boiling point byproducts at the top of the column and the excess of dichloropropanol and the heavy by-products at the bottom of the column. The top fraction, collected at a temperature of 60.8° C. under a pressure of 150 mbar, has been submitted continuously to a second distillation in a packed column to eliminate water and the low boiling by-products. The bottom product has been collected at normal pressure at a temperature of 115.2° C.; it contained 0.35 g of 1,3-dichloropropane per kg. This product has been purified by a third operation of distillation that has been realized in a batchwise manner in a glass plate distillation column under vacuum at a pressure of 790 mbar. The epichlorohydrin which has been recovered as the main distillation fraction collected between 107.4-107.6° C. contained 0.34 g of 1,3-dichloropropane per kg and is named ECH 2.

### EXAMPLE 4

A well stirred reactor has been fed continuously with dichloropropanol containing 0.013 g of 1,3-dichloropropane and 0.0026 g of 3-chloro-1-propanol per kg of dichloropropanol at a rate of 5.93 kg/h/l of reactor, with a sodium hydroxide solution containing 500 g of NaOH per kg at rate of 2.67 kg/h/l of reactor and with recycled water at a rate of 5.83 kg/h/l of reactor. The reactor has been operated at a temperature of 45° C. and at a pressure of 1 bar. The reactor was fitted with an overflow system for withdrawal of the liquid reaction medium. The liquid reaction medium was biphasic. The lighter phase which contained the salt and a small quantity of organics has been stripped with steam and the condensate has been recycled to the reactor. The heavier phase which contained the major part of the epichlorohydrin formed in the reactor has been submitted continuously to a first distillation operation in a packed column to separate epichlorohydrin, water and the low boiling point byproducts at the top of the column and the excess of dichloropropanol and the heavy by-products at the bottom of the column. The top fraction, collected at a temperature of 62.4° C. under a pressure of 150 mbar, has been submitted continuously to a second distillation in a packed column to eliminate water and the low boiling by-products as the top product and some heavy impurities at the bottom of the column. The epichlorohydrin has been collected by condensation of a gas phase collected from the middle of the column at normal pressure and at a temperature of 116.4° C.; it contained 0.03 g of 1,3-dichloropropane per kg and is named ECH 5.

The complete composition of epichlorohydrins ECH 2 and ECH 5 and of three other epichlorohydrin samples (ECH 1, ECH 3 and ECH 4) are presented in Table 1.

Their compositions obtained by gas chromatography analysis.

TABLE 1

| Component | | | | | |
|---|---|---|---|---|---|
| (g/kg) | ECH 1 | ECH 2 | ECH 3 | ECH 4 | ECH 5 |
| Acetaldehyde | 0.004 | n.d | n.d | n.d. | n.d. |
| Acrolein | <0.001 | 0.003 | 0.003 | n.d. | n.d. |
| 2-propanol | <0.001 | n.d. | n.d. | n.d. | n.d. |
| 3-chloro-1-propene | n.d. | n.d. | n.d. | n.d. | n.d. |
| allyl alcohol | 0.001 | <0.001 | <0.001 | n.d. | 0.003 |
| Hydroxyacetone | 0.094 | 0.018 | 0.018 | 0.006 | 0.006 |
| chloroacetone + (3,3- | 0.033 | 0.038 | 0.040 | n.d. | 0.024 |
| dichloro-1-propene) | | | | | |
| 1,2-dichloropropane | 0.042 | n.d. | n.d. | 0.001 | n.d. |
| 2,3-dichloro-1-propene | 0.005 | n.d. | n.d. | 0.004 | n.d. |
| 1-chloro-2,3- | >998.464 | >999.474 | >999.045 | >999.503 | >999.865 |
| epoxypropane (*) | | | | | |
| 1,3-dichloro-1-propene | 0.219 | 0.008 | 0.008 | 0.032 | 0.004 |
| cis maj. + (C6H14O min.) | | | | | |
| 2-chloro-2-propene-1-ol | 0.348 | 0.016 | 0.016 | 0.14 | 0.012 |
| 1,3-dichloro-1-propene | 0.035 | 0.010 | 0.010 | 0.008 | 0.009 |
| trans | | | | | |
| C₅H₁₀O/C₄H₇ClO | n.d. | n.d. | n.d. | 0.014 | 0.001 |
| C₆H₁₂O | n.d. | n.d. | n.d. | 0.011 | <0.001 |
| 1,3-dichloropropane | 0.002 | 0.34 | 0.34 | 0.005 | 0.030 |
| Cyclopentanone | 0.001 | 0.004 | 0.004 | n.d. | 0.004 |
| dibromochloromethane | 0.004 | n.d. | n.d. | 0.084 | n.d. |
| C₆H₁₀O iso 1 | 0.003 | n.d. | n.d. | 0.009 | <0.001 |
| C₆H₁₀O iso 2 | 0.012 | n.d. | n.d. | 0.009 | 0.001 |
| 1,2-epoxyhexane + (1,2,2- | 0.030 | 0.002 | 0.002 | n.d. | 0.001 |
| trichloropropane) | | | | | |
| C₆H₁₀O iso 3 | 0.004 | n.d. | n.d. | 0.031 | 0.001 |
| dichloroepoxypropane + | 0.003 | n.d. | n.d. | 0.006 | n.d. |
| Ni | | | | | |
| 1,3,3-trichloro-1-propene | 0.012 | n.d. | n.d. | 0.004 | n.d. |
| cis + 1,1,3- | | | | | |
| trichloropropene | | | | | |
| 1,1,2-trichloropropane | 0.211 | 0.001 | 0.001 | 0.025 | 0.007 |
| chlorobenzene | 0.011 | <0.001 | <0.001 | 0.001 | 0.007 |
| 1,3,3-trichloro-1-propene | 0.015 | n.d. | n.d. | 0.012 | 0.001 |
| trans | | | | | |
| 1,2,3-trichloropropene | 0.016 | <0.001 | <0.001 | 0.003 | 0.001 |
| trans | | | | | |
| 1,3-dichloro-2-propanol | 0.111 | 0.023 | 0.024 | 0.017 | 0.008 |
| 1,2,3-trichloropropane | 0.014 | n.d. | n.d. | 0.024 | n.d. |
| 1,2,3-trichloropropene cis | 0.002 | n.d. | n.d. | n.d. | n.d. |
| 3-chloro-1,2-propanediol + | 0.13 | <0.001 | 0.001 | n.d. | 0.001 |
| 2,3-dichloro-1-propanol | | | | | |
| C₆H₁₃Br | n.d. | n.d. | n.d. | 0.005 | n.d. |
| C₆H₁₀Cl₂ iso 1 | n.d. | n.d. | n.d. | 0.005 | n.d. |
| C₆H₁₀Cl₂ iso 2 | n.d. | n.d. | n.d. | 0.004 | n.d. |
| methyl glycidyl ether | 0.007 | 0.054 | 0.48 | n.m. | n.m. |
| Unknowns (sum) | 0.170 | 0.007 | 0.008 | 0.087 | 0.024 |

n.d.: not detected,
n.m.: not measured
(*) 1-chloro-2,3-epoxypropane amount calculated on the basis of the total content of other organic components

### EXAMPLES 5 to 14 (Homopolymerization of ECH)

The tests have been carried out according to the following procedure with epichlorohydrin sample ECH1 (examples 5 to 7), ECH 2 (examples 8 to 10) and ECH 3 (examples 11 to 14). The quantities of chemicals are indicated in Table 2.

The polymerization of epichlorohydrin (ECH) has been carried out in the presence of the system tetraoctylammonium bromide (Noct₄Br)/triisobutyl aluminium (TiBA).

The epichlorohydrin has been dried over calcium hydride under vacuum for 24 h at 25° C. and further distilled.

The polymerization reactions have been carried out in pyrex vessels fitted with polytetrafluorethylene valves. The vessels have been evacuated under flame heating to remove residual moisture. After cooling to room temperature, the vessels have been cooled to −30° C. (ethanol/liquid nitrogen cooling bath) and toluene and epichlorohydrin, have been added under vacuum. After those additions, argon has been introduced in the vessel and tetraoctylammonium bromide and triisobutyl aluminium have been added to the vessel. This addition constituted the time zero of the reaction. After a given time under magnetic stirring at −30° C., the reaction has been stopped by adding 1-2 ml of ethanol to the vessel. Half of the volume of the reaction medium has then been submitted to evaporation after which the polymer has been recovered from the vessel.

The conversion has been obtained by comparing the weight of recovered polymer with the weight of added epichlorohydrin.

The theoretical molar weight (Mn th.) has been calculated on the basis of the quantity of tetraoctylammonium bromide.

The measured polymer molar weight (Mn exp) and the molar weight dispersion have been obtained by Gel Permeation Chromatography.

The tacticity of the polymer has been obtained by ¹³C and ¹H NMR.

The results of the tests are summarized in Table 3.

TABLE 2

| Example n^{o} | ECH (ml) | Toluene (ml) | Noct₄Br (ml) | TiBA (ml) |
|---|---|---|---|---|
| 5 | 4 | 10.2 | 2.15 | 0.71 |
| 6 | 4 | 10.2 | 2.15 | 0.71 |
| 7 | 3.4 | 9.9 | 0.91 | 0.30 |
| 8 | 4 | 10.2 | 2.15 | 0.71 |
| 9 | 4 | 10.2 | 2.15 | 0.71 |
| 10 | 4 | 11.6 | 1.08 | 0.35 |
| 11 | 4 | 10.2 | 2.15 | 0.71 |
| 12 | 4 | 10.2 | 2.15 | 0.71 |
| 13 | 3.6 | 11.4 | 0.97 | 0.43 |
| 14 | 4 | 11.6 | 1.08 | 0.35 |

TABLE 3

| | Reaction | Conversion | Mn th. | Mn exp. | | |
|---|---|---|---|---|---|---|
| Example | time (h) | (mol %) | (g/mol) | (g/mol) | Dispersion | Tacticity |
| 5 | 1 | 100 | 10000 | 10700 | 1.17 | atactic |
| 6 | 1 | 100 | 10000 | 10100 | 1.23 | n.m. |
| 7 | 2 | 100 | 20000 | 20200 | 1.17 | n.m. |
| 8 | 1 | 100 | 10000 | 16400 | 1.22 | n.m. |
| 9 | 1 | 100 | 10000 | 11200 | 1.20 | atactic |
| 10 | 1 | 100 | 20000 | 77700 (20%) | | n.m. |
| | | | | 22200 (80%) | | |
| 11 | 1 | 80 | 8000 | 6800 | 1.17 | n.m. |
| 12 | 2 | 95 | 9500 | 12100 | 1.17 | atactic |
| 13 | 2 | 90 | 18000 | 24700 | 1.18 | n.m. |
| 14 | 6 | 94 | 18800 | 17650 | 1.17 | n.m. |

n.m.: not measured

### EXAMPLES 15 to 17 (Homopolymerization of ECH)

The tests have been carried out according to the following procedure with epichlorohydrin sample ECH1 (example 15), ECH 2 (example 16) and ECH 3 (example 17). The quantities of chemicals are indicated in Table 4.

The polymerization of epichlorohydrin (ECH) has been carried out in the presence of the system water/triethyl aluminium (TEA).

The procedure of example 1 has been followed except that TEA in solution in toluene and water have been added under argon to the vessel first evacuated and dried, left under magnetic stirring under vacuum for 30 min, before ECH in toluene has been added (time zero of the reaction). The polymerization has been carried out at a temperature of 25° C. for 12 h. The results have been summarized in Table 5

TABLE 4

| Example n^{o} | ECH (ml) | Toluene (ml) | H20 (μl) | TEA (ml) |
|---|---|---|---|---|
| 15 | 4 | 10 | 23 | 0.67 |
| 16 | 4 | 10 | 23 | 0.67 |
| 17 | 4 | 10 | 23 | 0.67 |

TABLE 5

| | Reaction | Conversion | Mn exp. | | |
|---|---|---|---|---|---|
| Example | time (h) | (mol %) | (g/mol) | Dispersion | Tacticity |
| 15 | 12 | 47 | 216000 | 2.02 | n.m. |
| | | | 7000 | 1.04 | |
| 16 | 12 | 50 | 285200 | 3.51 | atactic |
| | | | 5850 | 1.08 | |
| 17 | 12 | 55 | 357600 | 3.45 | atactic |
| | | | 8100 | 1.31 | |

n.m.: not measured

### EXAMPLE 18

Preparation of a product consisting predominantly in diglycidyl diether of Bisphenol A according to U.S. Pat. No. 2,811,227

The apparatus employed was a thermostatised flask equipped with a mechanical stirrer, with a jacket containing a thermocouple and with a Dean-Stark separator surmounted by a water-cooled condenser. A pump was used to inject a caustic soda aqueous solution at a constant rate in the flask.

The reaction flask was initially charged with a mixture of bisphenol A (68.4 g, 0.3 mol) and the epichlorohydrin sample ECH4 coming from a propylene-chlorine plant (277.5 g, 3.0 mol). The analysis of the epichlorydrin is given in Table 1. The mixture was heated at reflux under stiffing to a temperature of 111° C. A 40% aqueous solution of caustic soda (60.8 g, 0.6 mol) was introduced at a rate of 12 ml/h during 3.5 hour. The temperature of the mixture in the flask was maintained in the range 100° C.-115° C. in order to assure a constant reflux. The epichlorohydrin rich organic phase decanted during the reaction as a lower phase in the separator was recycled regularly in the reaction flask and the aqueous rich phase collected as an upper phase in the separator was regularly drawn off. The heating was maintained for 15 min after the total introduction of the caustic soda solution to achieve the collect of the water phase in the decantor. 29.7 g of aqueous phase was collected with a composition given in Table 6.

The epichlorohydrin in excess was removed from the reaction mixture by distillation under a vacuum of 30 mbar and by a progressive heating of the mixture to 109° C. 156.1 g (1.7 mol) of epichlorohydrin was recovered in this step. The composition of the distillate is given in Table 6.

The salt was separated from the crude product (45.5 g) after addition of 567.2 g of toluene under agitation and by filtration. The cake of filtration was washed with 124.4 g of toluene. The toluene solutions were mixed and evaporated at 185° C. under a pressure of 1 mbar.

659.4 g of toluene was recovered as the condensate of the evaporated fraction with a composition given in Table 6. The residual product of the evaporation (100.5 g) contained the diglycidyl ether of bis-phenol A as a major product and no trace of unconverted bis-phenol A (<5 mg/kg). The residue contained 4.98 mol epoxy per kg and 1.52% of hydrolysable chlorine.

### EXAMPLE 19

The trial was realized in the apparatus described in example 18.

The reaction flask was initially charged with a mixture of bisphenol A (68.4 g, 0.3 mol) and epichlorohydrin sample ECH 5 (277.5 g, 3.0 mol). The analysis of the epichlorydrin is given in Table 1. The mixture was heated at reflux under stiffing to a temperature of 119° C. A 40% aqueous solution of caustic soda (60.8 g, 0.6 mol) was introduced at a rate of 12 ml/h during 3.5 hour. The temperature of the mixture in the flask was maintained in the range 102° C.-119° C. in order to assure a constant reflux. The epichlorohydrin rich organic phase decanted during the reaction as a lower phase in the separator was recycled regularly in the reaction flask and the aqueous rich phase collected as an upper phase in the separator was regularly drawn off. The heating was maintained for 15 min after the total introduction of the caustic soda solution to achieve the collect of the water phase in the decantor. 54.5 g of aqueous phase was collected with a composition given in Table 6.

The epichlorohydrin in excess was removed from the reaction mixture by distillation under a vacuum of 30 mbar and by a progressive heating of the mixture to 118° C. 148.2 g (1.5 mol) of epichlorohydrin was recovered in this step. The composition of the distillate is given in Table 6.

The salt was separated from the crude product (47.8 g) after addition of 228.4 g of toluene under agitation and by filtration. The cake of filtration was washed with 97.3 g of toluene. The toluene solutions were mixed and evaporated at 180° C. under a pressure of 1 mbar.

305.0 g of toluene was recovered as the condensate of the evaporation with a composition given in Table 6. The residual product of the evaporation (99.8 g) contained the diglycidyl ether of bis-phenol A as a major product and no trace of unconverted bis-phenol A (<5 mg/kg). The residue contained 4.93 mol epoxy per kg and 0.49% of hydrolysable chlorine.

The High Performance Liquid Chromatography analyses of the residual products obtained in examples 18 and 19 are similar.

TABLE 6

| | Example 18 | | | Example 19 | | |
|---|---|---|---|---|---|---|
| | Epichlorohydrin | Water | Toluene | Epichlorohydrin | Water | Toluene |
| | evaporated | evaporated | evaporated | evaporated | evaporated | evaporated |
| Component | (g/kg) | (mg/l) | (g/kg) | (g/kg) | (mg/l) | (g/kg) |
| acetaldehyde | n.d. | 2.9 | n.d. | n.d. | 1.3 | n.d. |
| acrolein | n.d. | 0.58 | n.d. | 0.002 | 0.42 | n.d. |
| 2-propanol | n.d. | <0.05 | n.d. | n.d. | 0.3 | n.d. |
| 3-chloro-1-propene | 0.001 | n.d. | n.d. | n.d. | n.d. | n.d. |
| allyl alcohol | n.d. | n.d. | n.d. | 0.001 | 0.2 | n.d. |
| hydroxyacetone | 0.016 | n.d. | n.d. | 0.002 | n.d. | n.d. |
| chloroacetone + (3,3- | 0.003 | 0.65 | n.d. | 0.002 | 0.53 | n.d. |
| dichloro-1-propene) | | | | | | |
| 1,2-dichloropropane | | | n.d. | n.d. | n.d. | n.d. |
| 2,3-dichloro-1-propene | 0.005 | 0.07 | n.d. | n.d. | n.d. | n.d. |
| 1-chloro-2,3-epoxypropane | principal | (45 g/kg) | 1.6 | principal | (46 g/kg) | 3.3 |
| | product | | | product | | |
| 1,3-dichloro-1-propene cis | 0.026 | 0.36 | n.d. | 0.003 | n.d. | n.d. |
| maj. + (C6H14O min.) | | | | | | |
| 2-chloro-2-propene-1-ol | 0.19 | 0.12 | n.d. | 0.016 | <0.05 | n.d. |
| 1,3-dichloro-1-propene trans | 0.007 | <0.05 | n.d. | 0.008 | n.d. | n.d. |
| C₅H₁₀O/C₄H₇ClO | 0.019 | 0.05 | n.d. | 0.001 | n.d. | n.d. |
| C₆H₁₂O | 0.022 | 0.28 | n.d. | 0.021 | n.d. | n.d. |
| 1,3-dichloropropane | 0.001 | n.d. | n.d. | 0.03 | <0.05 | n.d. |
| Cyclopentanone | n.d. | n.d. | n.d. | 0.006 | n.d. | n.d. |
| dibromochloromethane | 0.080 | n.d. | n.d. | n.d. | n.d. | n.d. |
| C₆H₁₀O iso 1 | 0.033 | 0.11 | n.d. | 0.038 | n.d. | n.d. |
| C₆H₁₀O iso 2 | 0.040 | 0.31 | n.d. | 0.001 | n.d. | n.d. |
| 1,2-epoxyhexane + (1,2,2- | n.d. | n.d. | n.d. | 0.001 | n.d. | n.d. |
| trichloropropane) | | | | | | |
| C₆H₁₀O iso 3 | 0.036 | 0.21 | n.d. | 0.002 | n.d. | n.d. |
| dichloroepoxypropane | 0.006 | | n.d. | n.d. | n.d. | n.d. |
| 1,3,3-trichloro-1-propene cis + | 0.006 | n.d. | n.d. | n.d. | n.d. | n.d. |
| 1,1,3-trichloropropene | | | | | | |
| 1,1,2-trichloropropane | 0.005 | n.d. | n.d. | | n.d. | n.d. |
| chlorobenzene | 0.001 | n.d. | n.d. | 0.008 | n.d. | n.d. |
| 1,3,3-trichloro-1-propene | 0.009 | n.d. | n.d. | n.d. | n.d. | n.d. |
| trans | | | | | | |
| 1,2,3-trichloropropene trans | 0.003 | n.d. | n.d. | 0.001 | n.d. | n.d. |
| 1,3-dichloro-2-propanol | 3.4 | 143 | 0.38 | 2.5 | 111 | 0.074 |
| 1,2,3-trichloropropane | 0.022 | n.d. | 0.002 | n.d. | n.d. | n.d. |
| 1,2,3-trichloropropene cis | n.d. | n.d. | n.d. | n.d. | n.d. | n.d. |
| 3-chloro-1,2-propanediol + | 0.13 | 5.9 | 0.064 | 0.071 | 4.1 | 0.033 |
| 2,3-dichloro-1-propanol | | | | | | |
| C₆H₁₃Br | n.d. | <0.05 | 0.005 | n.d. | <0.05 | |
| C₆H₁₀Cl₂ iso 1 | 0.009 | n.d. | n.d. | n.d. | n.d. | n.d. |
| C₆H₁₀Cl₂ iso 2 | 0.007 | n.d. | n.d. | n.d. | n.d. | n.d. |
| methyl glycidyl ether | n.m. | n.m. | n.m. | n.m. | n.m. | n.m. |
| Unknowns (sum) | 0.299 | 10.00 | 1.31 | 0.213 | 1.3 | 1.373 |

n.d.: not detected,
n.m.: not measured

1. A process for manufacturing dichloropropanol wherein a glycerol-based product comprising at least one diol containing at least 3 carbon atoms other than 1,2-propanediol, is reacted with a chlorinating agent.

2. The process according to claim 1, wherein the diol is selected from the group consisting of 1,3-propanediol, butanediol, and mixtures thereof.

3. The process according to claim 1, wherein the glycerol-based product comprises, in addition, 1,2-ethanediol and/or 1,2-propanediol.

4. The process according to claim 1, wherein the glycerol-based product comprises, in addition, at least one compound selected from the group consisting of glycerol alkyl ethers, monoalcohols, water, ketones, aldehydes, alkyl esters of fatty acids, glycerol esters, carboxylic acids, glycerol oligomers, salts, compounds containing nitrogen, and any mixture of at least two of them.

5. The process according to claim 1, wherein the glycerol content of the glycerol-based product is greater than or equal to 500 g/kg of product, wherein the content of diol containing at least 3 carbon atoms other than 1,2-propanediol of the glycerol-based product is greater than or equal to 0.001 g/kg of product and less than or equal to 100 g/kg of product, and wherein the content of 1,2-ethanediol and/or 1,2-propanediol of the glycerol-based product, when present, is greater than or equal to 0.001 g/kg of product and less than or equal to 100 g/kg of product.

6. The process according to claim 1, wherein the glycerol-based product is subjected to at least one treatment, optionally under reduced pressure, selected from the group consisting of evaporative concentration, evaporative crystallization, distillation, fractional distillation, stripping, and liquid/liquid extraction, in order to obtain a purified glycerol-based product, prior to the reaction with the chlorinating agent.

7. The process according to claim 6, wherein the purified glycerol-based product comprises at least one diol containing at least 3 carbon atoms other than 1,2-propanediol, in an amount less than 0.7 g diol/kg of purified product.

8. The process according to claim 1, wherein the chlorinating agent comprises hydrogen chloride.

9. A process for manufacturing epichlorohydrin, wherein dichloropropanol, at least one part of which is obtained according to the process of claim 1, is subjected to a dehydrochlorination reaction.

10. A process for manufacturing a product selected from the group consisting of epoxy resins, glycidyl esters, glycidyl ethers, glycidyl amides, glycidyl imides, coagulants, wet-strength resins, cationization agents, flame retardants, detergent ingredient, and epichlorohydrin elastomers, wherein epichlorohydrin, at least one part of which is obtained according to the process of claim 9, is subjected to a reaction with at least one compound containing at least one active hydrogen atom.

11. A method for manufacturing dichloropropanol comprising utilizing a glycerol-based product comprising at least one diol containing at least 3 carbon atoms other than 1,2-propanediol.

12. A glycerol-based product that comprises at least one diol containing at least 3 carbon atoms other than 1,2-propanediol.

13. The glycerol-based product according to claim 12 wherein the diol is in an amount of less than 0.7 g of diol/kg of product.

14. The glycerol-based product according to claim 12 wherein the diol is in an amount of less than 0.5 g of diol/kg of product.
